(19) United States
(12) Patent Application Publication
(10) Pub. No.: US 2025/0204925 A1
(43) Pub. Date: Jun. 26, 2025

(54) GAP SEALING SYSTEM AND KIT

(71) Applicant: BOSTON SCIENTIFIC SCIMED, INC., MAPLE GROVE, MN (US)

(72) Inventors: Patrick Willoughby, Shoreview, MN (US); Scott Andrew McCullough, Plymouth, MN (US); Emily Vance, Minneapolis, MN (US)

(73) Assignee: BOSTON SCIENTIFIC SCIMED, INC., MAPLE GROVE, MN (US)

(21) Appl. No.: 18/987,281

(22) Filed: Dec. 19, 2024

Related U.S. Application Data
(60) Provisional application No. 63/612,493, filed on Dec. 20, 2023.
(60) Provisional application No. 63/612,507, filed on Dec. 20, 2023.
(60) Provisional application No. 63/612,569, filed on Dec. 20, 2023.
(60) Provisional application No. 63/612,582, filed on Dec. 20, 2023.
(60) Provisional application No. 63/561,406, filed on Mar. 5, 2024.
(60) Provisional application No. 63/561,415, filed on Mar. 5, 2024.
(60) Provisional application No. 63/560,160, filed on Mar. 1, 2024.
(60) Provisional application No. 63/560,174, filed on Mar. 1, 2024.

(51) Int. Cl. A61B 17/12; A61B 17/00; A61B 90/00

(52) U.S. Cl. CPC A61B 17/12172; A61B 17/12031; A61B 17/12122; A61B 90/39; A61B 2017/00477; A61B 2017/00862; A61B 2017/00871; A61B 2017/1205; A61B 2090/3966

(57) ABSTRACT

A gap sealing system may include a piece of expandable foam configured to expand in vivo, and an anchoring element extending from the piece of expandable foam. The anchoring element may include at least one arm extending distally from the foam. The foam may be configured to be disposed proximal of an occlusive implant to occlude an ostium of the left atrial appendage. The at least one arm may be configured to extend into an interior of the implant in vivo to anchor the foam to the implant. A kit may include the piece of expandable foam, a second piece of expandable foam configured to expand in vivo, wherein the second piece of expandable foam has an overall volume different from the piece of expandable foam, a tubular member configured to deliver the piece of expandable foam, and a second tubular member configured to deliver the second piece of expandable foam.

### CROSS-REFERENCE TO RELATED APPLICATIONS

This application claims the benefit of priority of U.S. Provisional Application No. 63/612,493 filed Dec. 20, 2023, 63/612,507, filed Dec. 20, 2023, 63/612,569, filed Dec. 20, 2023, 63/612,582, filed Dec. 20, 2023, 63/561,406, filed Mar. 5, 2024, 63/561,415, filed Mar. 5, 2024, 63/560,160, filed Mar. 1, 2024, and 63/560,174, filed Mar. 1, 2024, the entirety disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. Existing left atrial appendage closure devices may leave leaks around the margins of the device, particularly in left atrial appendages have an irregular shape(s) and/or an irregular ostium. There is an ongoing need to provide alternative medical devices as well as alternative methods for manufacturing and using medical devices.

### SUMMARY

In one example, a gap sealing system for use with an occlusive implant disposed within a left atrial appendage of a patient may comprise a piece of expandable foam configured to expand from a delivery configuration to an expanded configuration in vivo, and an anchoring element extending from the piece of expandable foam.

In addition or alternatively to any example disclosed herein, the gap sealing system may comprise a radiopaque marker coupled to the piece of expandable foam.

In addition or alternatively to any example disclosed herein, the anchoring element is fixedly attached to the radiopaque marker.

In addition or alternatively to any example disclosed herein, the anchoring element is integrally formed with the piece of expandable foam.

In addition or alternatively to any example disclosed herein, the anchoring element is formed from a different material than the piece of expandable foam.

In addition or alternatively to any example disclosed herein, the anchoring element is formed from a metallic material.

In addition or alternatively to any example disclosed herein, the gap sealing system may comprise a tubular member sized and configured to deliver the piece of expandable foam to the left atrial appendage.

In addition or alternatively to any example disclosed herein, the anchoring element is configured to engage with the occlusive implant.

In addition or alternatively to any example disclosed herein, the anchoring element is configured to pierce tissue of the left atrial appendage.

In addition or alternatively to any example disclosed herein, the piece of expandable foam is formed from a shape memory polymer.

In addition or alternatively to any example disclosed herein, the piece of expandable foam is configured to be disposed alongside the occlusive implant to occlude a gap between the occlusive implant and a side wall of the left atrial appendage.

In addition or alternatively to any example disclosed herein, a gap sealing system for use with an occlusive implant disposed within a left atrial appendage of a patient may comprise a piece of expandable foam configured to expand from a delivery configuration to an expanded configuration in vivo, and an anchoring element comprising at least one arm extending distally from the piece of expandable foam in the expanded configuration, wherein the at least one arm comprises a longitudinally extending portion and a laterally extending portion. The piece of expandable foam may be configured to be disposed proximal of the occlusive implant to occlude an ostium of the left atrial appendage. The laterally extending portion of the at least one arm may be configured to extend into an interior of the occlusive implant in vivo to anchor the piece of expandable foam to the occlusive implant.

In addition or alternatively to any example disclosed herein, the laterally extending portion extends laterally from a distal end of the longitudinally extending portion.

In addition or alternatively to any example disclosed herein, the piece of expandable foam is not directly attachable to the occlusive implant.

In addition or alternatively to any example disclosed herein, a kit for use in sealing a gap with an occlusive implant disposed within a left atrial appendage of a patient may comprise a piece of expandable foam, wherein the piece of expandable foam is configured to expand from a delivery configuration to an expanded configuration in vivo, a second piece of expandable foam, wherein the second piece of expandable foam is configured to expand from a delivery configuration to an expanded configuration in vivo, wherein the piece of expandable foam has a first overall volume, and the second piece of expandable foam has a second overall volume different from the first overall volume, a tubular member sized and configured to deliver the piece of expandable foam to the left atrial appendage, and a second tubular member sized and configured to deliver the second piece of expandable foam to the left atrial appendage.

In addition or alternatively to any example disclosed herein, the piece of expandable foam and the second piece of expandable foam are each configured to be disposed alongside the occlusive implant to fill a gap between the occlusive implant and a side wall of the left atrial appendage.

In addition or alternatively to any example disclosed herein, the piece of expandable foam comprises an anchoring element extending distally from the piece of expandable foam, and the second piece of expandable foam comprises a second anchoring element extending distally from the second piece of expandable foam.

In addition or alternatively to any example disclosed herein, the anchoring element is formed from a shape memory polymer.

In addition or alternatively to any example disclosed herein, the anchoring element is formed from a metallic material.

In addition or alternatively to any example disclosed herein, the kit may comprise a third piece of expandable foam, wherein the third piece of expandable foam is configured to expand from a delivery configuration to an expanded configuration in vivo, the third piece of expandable foam having a third overall volume different from the first overall volume and the second overall volume, and a third tubular member sized and configured to deliver the third piece of expandable foam to the left atrial appendage.

The above summary of some embodiments, aspects, and/or examples is not intended to describe each embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 schematically illustrates selected aspects of a left atrial appendage;

FIG. 2 schematically illustrates selected aspects of an occlusive implant disposed within a left atrial appendage;

FIG. 3 schematically illustrates selected aspects of a gap sealing system and/or a kit for sealing a gap;

FIG. 4 schematically illustrates selected aspects of a gap sealing system for use with an occlusive implant disposed within a left atrial appendage;

FIG. 5 schematically illustrates selected aspects of a gap sealing system for use with an occlusive implant disposed within a left atrial appendage;

FIG. 6 schematically illustrates selected aspects of a gap sealing system for use with an occlusive implant disposed within a left atrial appendage;

FIG. 7 schematically illustrates selected aspects of a gap sealing system for use with an occlusive implant disposed within a left atrial appendage;

FIG. 8 schematically illustrates selected aspects of a gap sealing system for use with an occlusive implant disposed within a left atrial appendage; and

FIGS. 9-10 schematically illustrate selected aspects of a gap sealing system for use with an occlusive implant disposed within a left atrial appendage.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s).

Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

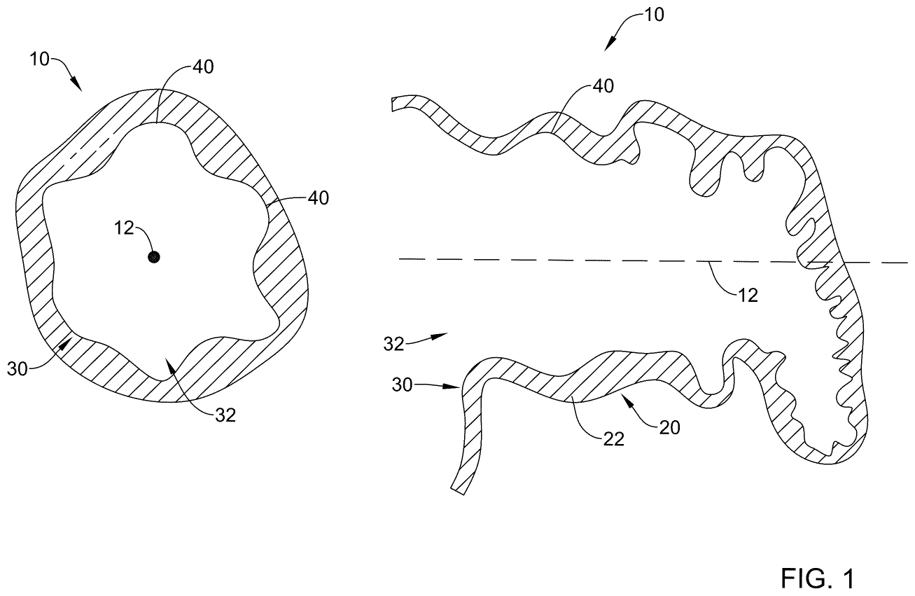

FIG. 1 schematically illustrates selected aspects of a left atrial appendage 10. It shall be understood that FIG. 1 is merely exemplary, and other geometries, shapes, sizes, etc. for the left atrial appendage 10 are common and may vary from patient to patient. The left atrial appendage 10 may be formed as a small pouch or extension attached to and extending from the left atrium of a patient's heart.

The left atrial appendage 10 may include a longitudinal axis 12 arranged along a depth of a main body 20 of the left atrial appendage 10. The main body 20 may include a side wall 22 and an ostium 30 forming a proximal mouth 32. In some embodiments, a lateral extent of the ostium 30 and/or the lateral wall 22 may be smaller or less than a depth of the main body 20 along the longitudinal axis 12, or a depth of the main body 20 may be greater than a lateral extent of the ostium 30 and/or the side wall 22. In some embodiments, the left atrial appendage 10 may narrow quickly along the depth of the main body 20 or the left atrial appendage 10 may maintain a generally constant lateral extent along a majority of depth of the main body 20.

The left atrial appendage 10 may comprise one or more lobes 40 formed in and/or by the side wall 22. In some embodiments, the one or more lobes 40 may comprise a widened area or portion of the main body 20. The one or more lobes 40 may be irregular in shape and/or size, and the shape and/or size may vary from lobe to lobe. In some embodiments, the one or more lobes 40 may be disposed at and/or adjacent to the ostium 30. In some embodiments, the one or more lobes 40 may be formed in and/or by the ostium 30, as viewed along the longitudinal axis 12.

In some embodiments, the left atrial appendage 10 may include a distalmost region formed or arranged as a tail-like element associated with a distal portion of the main body 20. In some embodiments, the distalmost region may protrude radially or laterally away from the longitudinal axis 12.

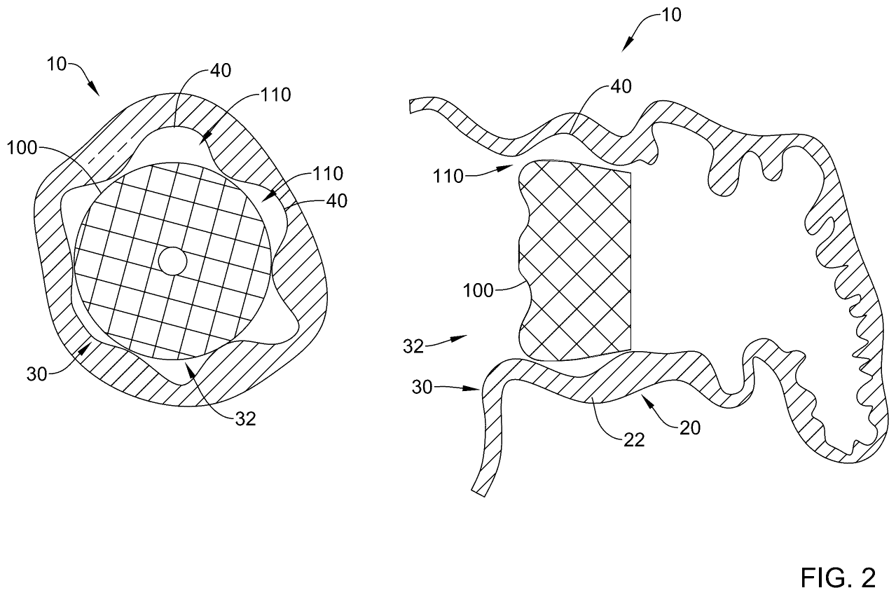

In FIG. 2, an occlusive implant 100 is shown disposed within the left atrial appendage 10. While many occlusive implants are generally round in shape, the occlusive implant 100 illustrated is merely exemplary, and other types, shapes, and/or sizes of occlusive implants may be used in its place. One example occlusive implant 100 that is commercially available is the WATCHMAN™ from Boston Scientific. Some occlusive implants may be designed and/or intended to stretch the ostium 30 and/or force the ostium and/or the left atrial appendage 10 to conform to the shape of the occlusive implant with an expandable framework that exerts a radially outward force against the left atrial appendage 10, the main body 20, and/or the ostium 30. However, in some cases, the left atrial appendage 10 and/or the ostium 30 may resist conforming, and/or may fail to fully conform, to the shape of the occlusive implant 100. Additionally, occlusive implants that are designed to be compliant, such that the occlusive implant 100 substantially conforms to the shape of the left atrial appendage 10 may be incapable of deforming sufficiently to completely close off the left atrial appendage 10 and/or the ostium 30. These phenomena may be more prevalent in left atrial appendages having one or more lobes 40 causing the ostium 30 and/or the main body 20 to have an irregular shape and/or a shape that is more irregular than what is considered “normal”.

As such, in some embodiments, the occlusive implant 100 may fail to completely and/or properly close off the left atrial appendage 10, as seen in FIG. 2. In some examples, a gap 110 or gaps may form and/or may be formed around the margin(s) and/or the periphery of the occlusive implant 100. In some embodiments, the one or more lobes 40 may form the gap 110 or gaps around the margin(s) and/or the periphery of the occlusive implant 100. The gap 110 and/or gaps around the margin(s) and/or the periphery of the occlusive implant 100 may cause and/or result in leaks that could permit thrombus to escape from the left atrial appendage 10 into the left atrium and/or the patient's vasculature.

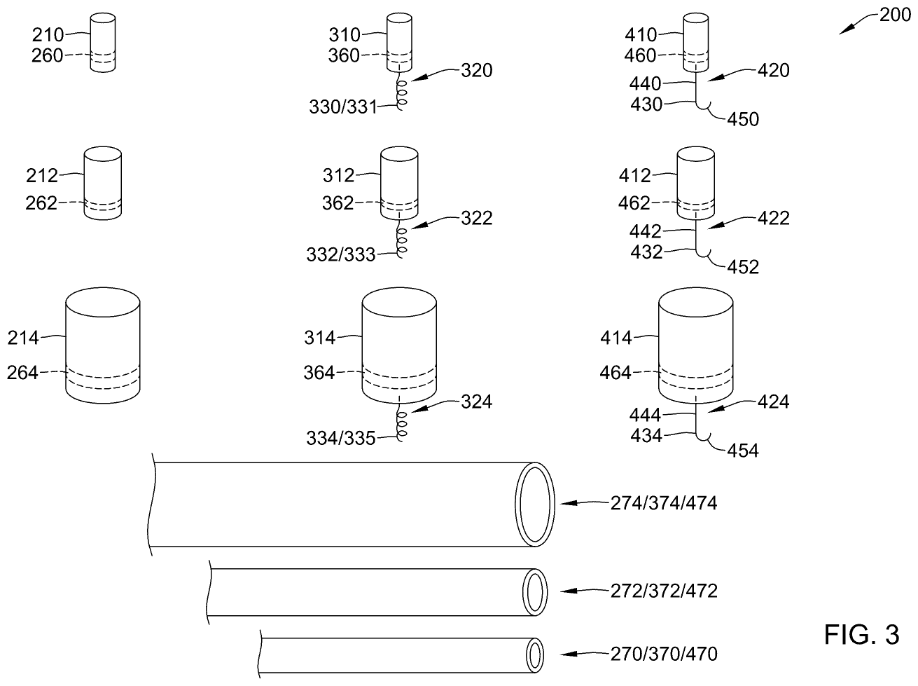

FIG. 3 schematically illustrates selected aspects of a gap sealing system 200 and/or a kit for use in sealing a gap 110 with an occlusive implant 100 disposed within the left atrial appendage 10. Various shapes, sizes, and configurations of the elements described herein may be included in the gap sealing system 200 and/or the kit. The elements and/or features of the gap sealing system 200 and/or the kit illustrated in FIG. 3 are not intended to be limiting and/or all-inclusive. The skilled person will recognize that the gap sealing system 200 and/or the kit may comprise fewer elements than those shown, including subsets of elements shown, or more elements than those shown in FIG. 3. Additionally, sizes, shapes, and/or quantities of elements may vary from system to system, kit to kit, within a system, and/or within a kit.

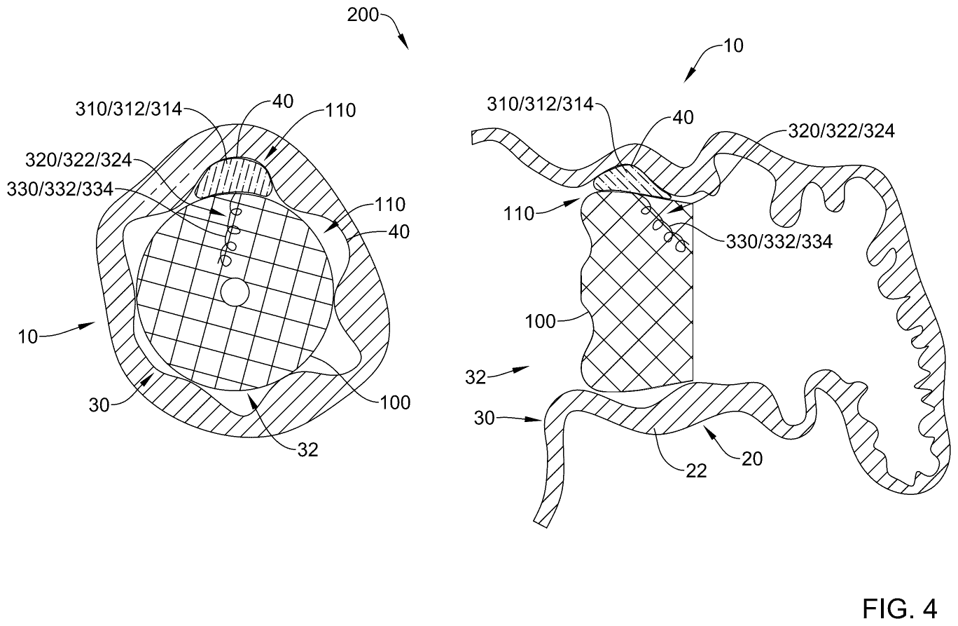

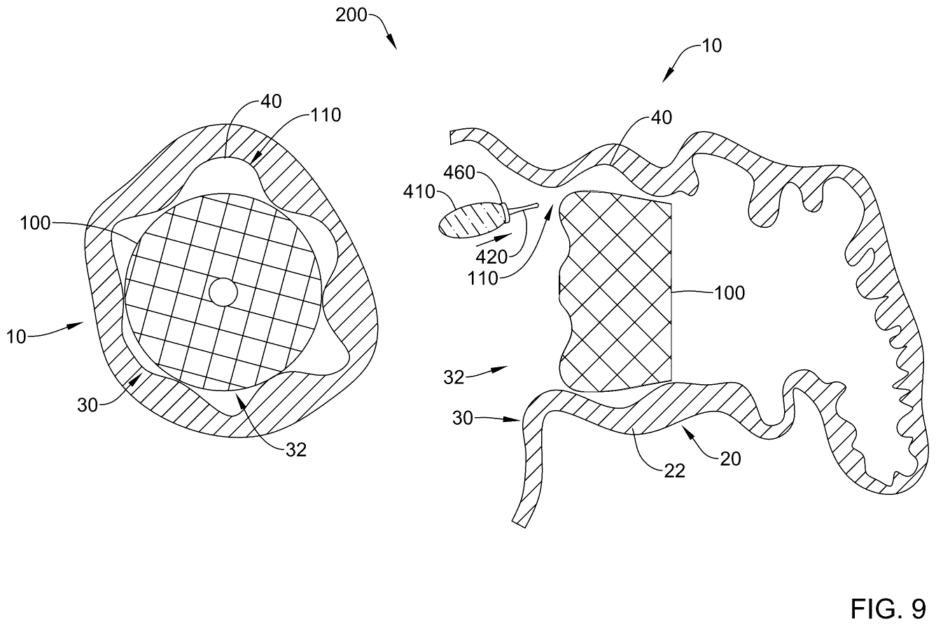

In some embodiments, the gap sealing system 200 and/or the kit may comprise a piece of expandable foam 210/310/410 configured to expand from a delivery configuration (e.g., FIGS. 3, 9) to an expanded configuration (e.g., FIGS. 4-8, 10) in vivo. In some embodiments, the gap sealing system 200 and/or the kit may comprise different sizes and/or shapes (e.g., different configurations) of the piece of expandable foam 210/310/410 which may be selected by the practitioner at the time of use and/or during an implantation procedure according to the desired usage.

In some embodiments, the gap sealing system 200 and/or the kit may comprise multiple instances of the piece of expandable foam 210/310/410 in a particular size and/or shape. In some embodiments, the gap sealing system 200 and/or the kit may comprise multiple instances of the piece of expandable foam 210/310/410 in different sizes and/or shapes. In some embodiments, where the one or more lobes 40 comprises two or more lobes, and/or the two or more lobes are different sizes, more than one piece of expandable foam 210/310/410 may be used and/or required.

In some embodiments, the gap sealing system 200 and/or the kit may comprise a piece of expandable foam 210 having a first configuration, a second piece of expandable foam 212 having the first configuration, and a third piece of expandable foam 214 having the first configuration, as seen in FIG. 3. In some embodiments, the second piece of expandable foam 212 may have a different size, a different shape, and/or a different configuration from the piece of expandable foam 210. In some embodiments, the third piece of expandable foam 214 may have a different size, a different shape, and/or a different configuration from the piece of expandable foam 210 and/or the second piece of expandable foam 212.

In some embodiments, the gap sealing system 200 and/or the kit may comprise a piece of expandable foam 310 having a second configuration, a second piece of expandable foam 312 having the second configuration, and a third piece of expandable foam 314 having the second configuration, as seen in FIG. 3. In some embodiments, the second piece of expandable foam 312 may have a different size, a different shape, and/or a different configuration from the piece of expandable foam 310. In some embodiments, the third piece of expandable foam 314 may have a different size, a different shape, and/or a different configuration from the piece of expandable foam 310 and/or the second piece of expandable foam 312.

In some embodiments, the gap sealing system 200 and/or the kit may comprise a piece of expandable foam 410 having a third configuration, a second piece of expandable foam 412 having the third configuration, and a third piece of expandable foam 414 having the third configuration, as seen in FIG. 3. In some embodiments, the second piece of expandable foam 412 may have a different size, a different shape, and/or a different configuration from the piece of expandable foam 410. In some embodiments, the third piece of expandable foam 414 may have a different size, a different shape, and/or a different configuration from the piece of expandable foam 410 and/or the second piece of expandable foam 412.

The piece of expandable foam 210/310/410 may be configured to expand from a delivery configuration (e.g., FIGS. 3, 9) to an expanded configuration (e.g., FIGS. 4-8, 10) in vivo. The second piece of expandable foam 212/312/412 may be configured to expand from a delivery configuration (e.g., FIGS. 3, 9) to an expanded configuration (e.g., FIGS. 4-8, 10) in vivo. The third piece of expandable foam 214/314/414 may be configured to expand from a delivery configuration (e.g., FIGS. 3, 9) to an expanded configuration (e.g., FIGS. 4-8, 10) in vivo.

In some embodiments, and in a non-limiting example, the piece of expandable foam 210/310/410, the second piece of expandable foam 212/312/412, and/or the third piece of expandable foam 214/314/414 may have a first generally cylindrical shape having a first radius in the delivery configuration and a second generally cylindrical shape having a second radius in the expanded configuration, wherein the second radius is greater than the first radius. The second generally cylindrical shape is maintained when the piece of expandable foam 210/310/410, the second piece of expandable foam 212/312/412, and/or the third piece of expandable foam 214/314/414 expands to the expanded configuration in a completely unconstrained environment. It will be understood that the piece of expandable foam 210/310/410, the second piece of expandable foam 212/312/412, and/or the third piece of expandable foam 214/314/414 may adapt to the shape and/or form of the gap 110, the occlusive implant 100, the left atrial appendage 10, etc. and thus may not form a perfectly cylindrical shape in the expanded configuration.

In one non-limiting example, the piece of expandable foam 210/310/410, the second piece of expandable foam 212/312/412, and/or the third piece of expandable foam 214/314/414 may have a second radius in the expanded configuration of about 3 millimeters, about 5 millimeters, about 8 millimeters, about 10 millimeters, about 15 millimeters, about 20 millimeters, etc. In use, the piece of expandable foam 210/310/410, the second piece of expandable foam 212/312/412, and/or the third piece of expandable foam 214/314/414 may be selected to fill and/or plug the gap 110 that is slightly smaller than the second radius in the expanded configuration such that the piece of expandable foam 210/310/410, the second piece of expandable foam 212/312/412, and/or the third piece of expandable foam 214/314/414 may be reasonably expected to completely fill the gap 110 in the expanded configuration. Other configurations, quantities, size, shapes, etc. are also contemplated.

In some embodiments, the piece of expandable foam 210/310/410 may have a first overall volume in the delivery configuration and/or the expanded configuration. In some embodiments, the second piece of expandable foam 212/312/412 may have a second overall volume in the delivery configuration and/or the expanded configuration different from the first overall volume. In some embodiments, the second overall volume may be about 10% different, about 20% different, about 30% different, about 40% different, about 50% different, about 75% different, about 100% different, about 125% different, about 150% different, about 200% different, etc. from the first overall volume. In at least some embodiments, the second overall volume may be greater than the first overall volume. In some embodiments, the second overall volume may be less than the first overall volume.

In some embodiments, the third piece of expandable foam 214/314/414 may have a third overall volume in the delivery configuration and/or the expanded configuration different from the first overall volume and the second overall volume. In some embodiments, the third overall volume may be about 10% different, about 20% different, about 30% different, about 40% different, about 50% different, about 75% different, about 100% different, about 125% different, about 150% different, about 200% different, etc. from the first overall volume and/or the second overall volume. In some embodiments, the third overall volume may be greater than the first overall volume. In some embodiments, the third overall volume may be less than the first overall volume. In some embodiments, the third overall volume may be greater than the second overall volume. In some embodiments, the third overall volume may be less than the second overall volume.

In at least some embodiments, the piece of expandable foam 210/310/410, the second piece of expandable foam 212/312/412, and/or the third piece of expandable foam 214/314/414 may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed or delivery size. In some examples, the collapsibility ratio of the shape memory polymer and/or the shape memory foam may be at least 5 times, at least 7 times, at least 8 times, at least 9 times, at least 10 times, at least 12 times, or more. In one example, a piece of expandable foam may have an outer diameter of about 32 millimeters in the expanded configuration and about 3 millimeters in the compressed configuration, producing a collapsibility ratio of at least 10 times (e.g., at least 10:1). Other configurations are also contemplated. In at least some embodiments, the piece of expandable foam 210/310/410, the second piece of expandable foam 212/312/412, and/or the third piece of expandable foam 214/314/414 may be configured as open celled foam.

In some embodiments, the piece of expandable foam 210/310/410, the second piece of expandable foam 212/312/412, and/or the third piece of expandable foam 214/314/414 and/or the shape memory foam may be formed from a biocompatible material. In at least some embodiments, the piece of expandable foam 210/310/410, the second piece of expandable foam 212/312/412, and/or the third piece of expandable foam 214/314/414 may be non-biodegradable and/or non-bioabsorbable. In some alternative embodiments, the piece of expandable foam 210/310/410, the second piece of expandable foam 212/312/412, and/or the third piece of expandable foam 214/314/414 may be biodegradable and/or bioabsorbable over time. In some embodiments, the piece of expandable foam 210/310/410, the second piece of expandable foam 212/312/412, and/or the third piece of expandable foam 214/314/414 may be configured to promote endothelization and/or tissue ingrowth. In some embodiments, the piece of expandable foam 210/310/410, the second piece of expandable foam 212/312/412, and/or the third piece of expandable foam 214/314/414 may include a coating, a material, and/or a component that promotes endothelization and/or tissue ingrowth. Other configurations are also contemplated.

In some embodiments, the piece of expandable foam 210/310/410, the second piece of expandable foam 212/312/412, and/or the third piece of expandable foam 214/314/414 may be configured to be biodegradable and/or bioabsorbable over time. In some embodiments, the piece of expandable foam 210/310/410, the second piece of expandable foam 212/312/412, and/or the third piece of expandable foam 214/314/414 may be configured to be biodegradable over at least 30 days′, 60 days′, 90 days′, 180 days′, or 365 days' time. Other configurations are also contemplated.

In some embodiments, the piece of expandable foam 210/310/410, the second piece of expandable foam 212/312/412, and/or the third piece of expandable foam 214/314/414 may be configured to prevent thrombus formation (e.g., within the left atrial appendage 10 and/or on the piece of expandable foam 210/310/410, the second piece of expandable foam 212/312/412, and/or the third piece of expandable foam 214/314/414). In some embodiments, the piece of expandable foam 210/310/410, the second piece of expandable foam 212/312/412, and/or the third piece of expandable foam 214/314/414 may include an anti-thrombus agent(s) and/or medicament(s). In some embodiments, the piece of expandable foam 210/310/410, the second piece of expandable foam 212/312/412, and/or the third piece of expandable foam 214/314/414 may be configured to absorb blood and/or bodily fluid(s). In some embodiments, the piece of expandable foam 210/310/410, the second piece of expandable foam 212/312/412, and/or the third piece of expandable foam 214/314/414 may be configured to trap thrombus. In some embodiments, the piece of expandable foam 210/310/410, the second piece of expandable foam 212/312/412, and/or the third piece of expandable foam 214/314/414 may be configured to promote tissue ingrowth and/or endothelization. Other configurations are also contemplated.

In some embodiments, the piece of expandable foam 210/310/410, the second piece of expandable foam 212/312/412, and/or the third piece of expandable foam 214/314/414 may each comprise different sizes and/or shapes in the delivery configuration and/or in the expanded configuration (when unconstrained). In some embodiments, the piece of expandable foam 210/310/410, the second piece of expandable foam 212/312/412, and/or the third piece of expandable foam 214/314/414 may be generally cylindrical, as shown in FIG. 3. In other examples, the piece of expandable foam 210/310/410, the second piece of expandable foam 212/312/412, and/or the third piece of expandable foam 214/314/414 may be oblong, cubic, spherical, or flattened, etc. Other configurations, including combinations thereof, are also contemplated.

In some embodiments, the piece of expandable foam 210/310/410, the second piece of expandable foam 212/312/412, and/or the third piece of expandable foam 214/314/414 may be unconstrained by outside forces and/or structure(s) in the delivery configuration. In some embodiments, the piece of expandable foam 210/310/410, the second piece of expandable foam 212/312/412, and/or the third piece of expandable foam 214/314/414 may be self-maintained in the delivery configuration by shape memory properties. Other configurations are also contemplated.

In some embodiments, the gap sealing system 200 and/or the kit may comprise an anchoring element 320/420 extending from the piece of expandable foam 310/410 in the delivery configuration and/or the expanded configuration. In some embodiments, the gap sealing system 200 may comprise a second anchoring element 322/422 extending from the second piece of expandable foam 312/412 in the delivery configuration and/or the expanded configuration. In some embodiments, the gap sealing system 200 may comprise a third anchoring element 324/424 extending from the third piece of expandable foam 314/414 in the delivery configuration and/or the expanded configuration.

In some embodiments, the anchoring element 320/420 may be fixedly attached to the piece of expandable foam 310/410. In some embodiments, the second anchoring element 322/422 may be fixedly attached to the second piece of expandable foam 312/412. In some embodiments, the third anchoring element 324/424 may be fixedly attached to the third piece of expandable foam 314/414. In some embodiments, the anchoring element 320/420 may be integrally formed with the piece of expandable foam 310/410. In some embodiments, the second anchoring element 322/422 may be integrally formed with the second piece of expandable foam 312/412. In some embodiments, the third anchoring element 324/424 may be integrally formed with the third piece of expandable foam 314/414. In some embodiments, the anchoring element 320/420, the second anchoring element 322/422, and/or the third anchoring element 324/424 may be formed from a metallic material, a polymeric material, or a shape memory material and/or a shape memory polymer. Other configurations are also contemplated.

In some embodiments, the anchoring element 320/420 may be formed from a different material than the piece of expandable foam 310/410. In some embodiments, the second anchoring element 322/422 may be formed from a different material than the second piece of expandable foam 312/412. In some embodiments, the third anchoring element 324/424 may be formed from a different material than the third piece of expandable foam 314/414.

In some embodiments, the anchoring element 320/420 may comprise at least one arm 330/430 extending distally from the piece of expandable foam 310/410 in the delivery configuration and/or the expanded configuration. In some embodiments, the at least one arm 430 and/or each arm of the at least one arm 430 may comprise a longitudinally extending portion 440 and a laterally extending portion 450. In some embodiments, the laterally extending portion 450 may extend laterally from a distal end of the longitudinally extending portion 440. In some embodiments, the longitudinally extending portion 440 and the laterally extending portion 450 may form a hook.

In some embodiments, the second anchoring element 422 may comprise at least one second arm 432 extending distally from the second piece of expandable foam 412 in the delivery configuration and/or the expanded configuration. In some embodiments, the at least one second arm 432 and/or each arm of the at least one second arm 432 may comprise a second longitudinally extending portion 442 and a second laterally extending portion 452. In some embodiments, the second laterally extending portion 452 may extend laterally from a distal end of the second longitudinally extending portion 442. In some embodiments, the second longitudinally extending portion 442 and the second laterally extending portion 452 may form a second hook.

In some embodiments, the third anchoring element 424 may comprise at least one third arm 434 extending distally from the third piece of expandable foam 414 in the delivery configuration and/or the expanded configuration. In some embodiments, the at least one third arm 434 and/or each arm of the at least one third arm 434 may comprise a third longitudinally extending portion 444 and a third laterally extending portion 454. In some embodiments, the third laterally extending portion 454 may extend laterally from a distal end of the third longitudinally extending portion 444. In some embodiments, the third longitudinally extending portion 444 and the third laterally extending portion 454 may form a third hook.

In some embodiments, the at least one arm 330 may comprise a helical coil 331 extending distally from the piece of expandable foam 310 in the delivery configuration and/or the expanded configuration, the at least one second arm 332 may comprise a second helical coil 333 extending distally from the second piece of expandable foam 312 in the delivery configuration and/or the expanded configuration, and the at least one third arm 334 may comprise a third helical coil 335 extending distally from the third piece of expandable foam 314 in the delivery configuration and/or the expanded configuration.

Other configurations, including combinations thereof, are also contemplated.

In some embodiments, the gap sealing system 200, the kit, and/or the piece of expandable foam 210/310/410 may optionally comprise a radiopaque marker 260/360/460 coupled to the piece of expandable foam 210/310/410. In some embodiments, the gap sealing system 200, the kit, and/or the second piece of expandable foam 212/312/412 may optionally comprise a second radiopaque marker 262/362/462 coupled to the second piece of expandable foam 212/312/412. In some embodiments, the gap sealing system 200, the kit, and/or the third piece of expandable foam 214/314/414 may optionally comprise a third radiopaque marker 264/364/464 coupled to the third piece of expandable foam 214/314/414.

In some embodiments, the anchoring element 320/420 may be fixedly attached to the radiopaque marker 360/460. In some embodiments, the second anchoring element 322/422 may be fixedly attached to the second radiopaque marker 362/462. In some embodiments, the third anchoring element 324/424 may be fixedly attached to the third radiopaque marker 364/464. Other configurations are also contemplated.

In some embodiments, the radiopaque marker 260/360/460 may be embedded within the piece of expandable foam 210/310/410. In some embodiments, the second radiopaque marker 262/362/462 may be embedded within the second piece of expandable foam 212/312/412. In some embodiments, the third radiopaque marker 264/364/464 may be embedded within the third piece of expandable foam 214/314/414. In some embodiments, the radiopaque marker 260/360/460 may comprise a radiopaque substance or a radiopaque material disposed within the piece of expandable foam 210/310/410 (e.g., the piece of expandable foam 210/310/410 may be doped with and/or may include the radiopaque substance or the radiopaque material). In some embodiments, the second radiopaque marker 262/362/462 may comprise a radiopaque substance or a radiopaque material disposed within the second piece of expandable foam 212/312/412 (e.g., the second piece of expandable foam 212/312/412 may be doped with and/or may include the radiopaque substance or the radiopaque material). In some embodiments, the third radiopaque marker 264/364/464 may comprise a radiopaque substance or a radiopaque material disposed within the third piece of expandable foam 214/314/414 (e.g., the third piece of expandable foam 214/314/414 may be doped with and/or may include the radiopaque substance or the radiopaque material).

In some embodiments, the gap sealing system 200 and/or the kit may comprise a tubular member 270/370/470 sized and configured to deliver the piece of expandable foam 210/310/410 to the left atrial appendage 10. In some embodiments, the gap sealing system 200 and/or the kit may comprise a second tubular member 272/372/472 sized and configured to deliver the second piece of expandable foam 212/312/412 to the left atrial appendage 10. In some embodiments, the gap sealing system 200 and/or the kit may comprise a third tubular member 274/374/474 sized and configured to deliver the third piece of expandable foam 214/314/414 to the left atrial appendage 10.

In some embodiments, the tubular member 270/370/470, the second tubular member 272/372/472, and/or the third tubular member 274/374/474 may be formed from a flexible material, such as a polymeric material. Other configurations are also contemplated. In some alternative embodiments, the tubular member 270/370/470, the second tubular member 272/372/472, and/or the third tubular member 274/374/474 may be a catheter, a delivery sheath, etc. Other configurations are also contemplated.

In some embodiments, during delivery to the left atrial appendage 10, the tubular member 270/370/470 may be adapted and configured to constrain the piece of expandable foam 210/310/410 in the delivery configuration. In some embodiments, during delivery to the left atrial appendage 10, the second tubular member 272/372/472 may be adapted and configured to constrain the second piece of expandable foam 212/312/412 in the delivery configuration. In some embodiments, during delivery to the left atrial appendage 10, the third tubular member 274/374/474 may be adapted and configured to constrain the third piece of expandable foam 214/314/414 in the delivery configuration.

In some embodiments, the gap sealing system 200 and/or the kit may comprise a push member (not shown) movably disposed within the tubular member 270/370/470, a second push member (not shown) movably disposed within the second tubular member 272/372/472, and/or a third push member (not shown) movably disposed within the third tubular member 274/374/474. In some embodiments, the push member may be configured to push and/or deploy the piece of expandable foam 210/310/410 out of the tubular member 270/370/470 into the gap 110. In some embodiments, the second push member may be configured to push and/or deploy the second piece of expandable foam 212/312/412 out of the second tubular member 272/372/472 into the gap 110. In some embodiments, the third push member may be configured to push and/or deploy the third piece of expandable foam 214/314/414 out of the third tubular member 274/374/474 into the gap 110. Other configurations and/or methods of deployment for the piece of expandable foam 210/310/410, the second piece of expandable foam 212/312/412, and/or the third piece of expandable foam 214/314/414 are also contemplated.

In some embodiments, the piece of expandable foam 210/310/410, the second piece of expandable foam 212/312/412, and/or the third piece of expandable foam 214/314/414 may be configured to shift from the delivery configuration toward and/or to the expanded configuration when unconstrained. In some embodiments, the piece of expandable foam 210/310/410, the second piece of expandable foam 212/312/412, and/or the third piece of expandable foam 214/314/414 may be configured to shift from the delivery configuration toward and/or to the expanded configuration when unconstrained by the tubular member 270/370/470, the second tubular member 272/372/472, and/or the third tubular member 274/374/474, respectively. In some embodiments, the piece of expandable foam 210/310/410, the second piece of expandable foam 212/312/412, and/or the third piece of expandable foam 214/314/414 may be configured to shift from the delivery configuration toward and/or to the expanded configuration under predetermined conditions (e.g., at a preselected temperature, when exposed to a preselected fluid or amount of fluid, etc.).

For the purpose of illustration, the gap sealing system 200 and/or the kit is illustrated in use in FIGS. 4-10 as including the anchoring element 320/420, the second anchoring element 322/422, and/or the third anchoring element 324/424. However, the skilled person will recognize that the piece of expandable foam 210, the second piece of expandable foam 212, and/or the third piece of expandable foam 214, which are devoid of an anchoring element, may be implanted in the same location(s) and/or for the same purpose(s).

In some embodiments, the piece of expandable foam 210/310/410, the second piece of expandable foam 212/312/412, and/or the third piece of expandable foam 214/314/414 may be configured to be disposed alongside the occlusive implant 100 (e.g., between the occlusive implant 100 and a side wall (e.g., the main body 20) of the left atrial appendage 10). In some embodiments, the piece of expandable foam 210/310/410, the second piece of expandable foam 212/312/412, and/or the third piece of expandable foam 214/314/414 may be configured to be disposed alongside the occlusive implant 100 to occlude and/or to fill a gap 110 or gaps around the margin(s) and/or the periphery of the occlusive implant 100 between the occlusive implant 100 and the side wall (e.g., the main body 20) of the left atrial appendage 10.

In some embodiments, the piece of expandable foam 210/310/410, the second piece of expandable foam 212/312/412, and/or the third piece of expandable foam 214/314/414 may be configured to adapt and conform to surrounding anatomy and/or structure(s) (e.g., the occlusive implant 100) when shifting from the delivery configuration toward and/or to the expanded configuration in vivo.

In some embodiments, the piece of expandable foam 210/310/410, the second piece of expandable foam 212/312/412, and/or the third piece of expandable foam 214/314/414 may be not directly attachable to the occlusive implant 100. In some embodiments, the piece of expandable foam 210/310/410, the second piece of expandable foam 212/312/412, and/or the third piece of expandable foam 214/314/414 may be configured to engage with and/or extend into an expandable framework of the occlusive implant 100. Other configurations are also contemplated.

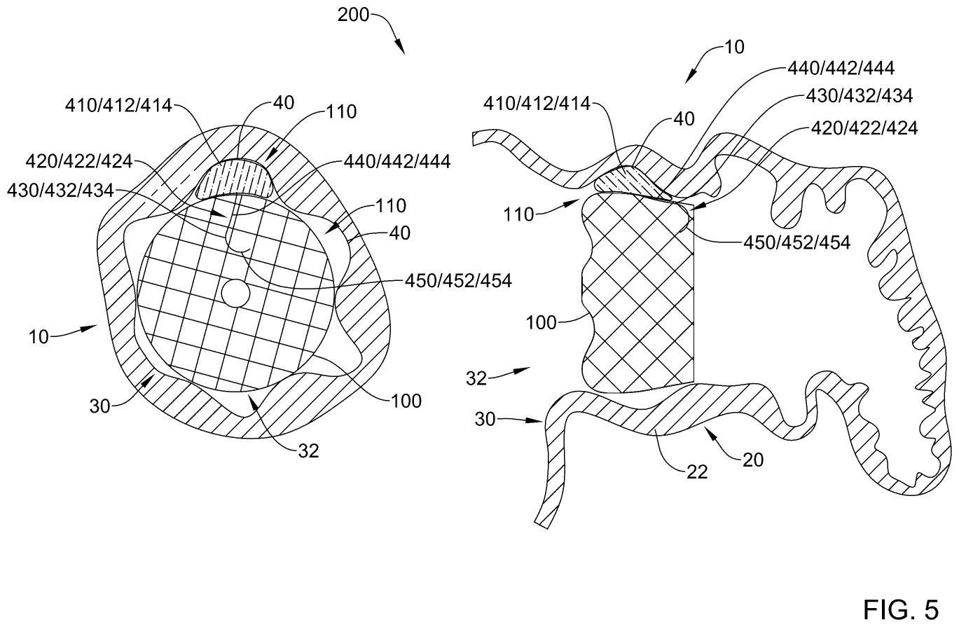

In some embodiments, the anchoring element 320/420, the second anchoring element 322/422, and/or the third anchoring element 324/424 may be configured to engage with the occlusive implant 100 and/or the expandable framework of the occlusive implant 100 within the left atrial appendage 10, as seen in FIGS. 4-5.

In some embodiments, the at least one arm 330/430, the at least one second arm 432, the at least one third arm 434 may be configured to engage with the occlusive implant 100 and/or the expandable framework of the occlusive implant 100 within the left atrial appendage 10. In some embodiments, the helical coil 331, the second helical coil 333, and/or the third helical coil 335 may be configured to engage with the occlusive implant 100 and/or the expandable framework of the occlusive implant 100 within the left atrial appendage 10, as seen in FIG. 4.

In some embodiments, the longitudinally extending portion 440 and/or the laterally extending portion 450, the second longitudinally extending portion 442 and/or the second laterally extending portion 452, and/or the third longitudinally extending portion 444 and/or the third laterally extending portion 454 may be configured to engage with the occlusive implant 100 and/or the expandable framework of the occlusive implant 100 within the left atrial appendage 10, as seen in FIG. 5. In some embodiments, the laterally extending portion 450 of the at least one arm 430, the second laterally extending portion 452 of the at least one second arm 432, and/or the third laterally extending portion 454 of the at least one third arm 434 may be configured to extend into an interior of the occlusive implant 100 and/or the expandable framework of the occlusive implant 100 in vivo to anchor the piece of expandable foam 410, the second piece of expandable foam 412, and/or the third piece of expandable foam 414 to the occlusive implant 100 within the left atrial appendage 10, as seen in FIG. 5. Other configurations are also contemplated.

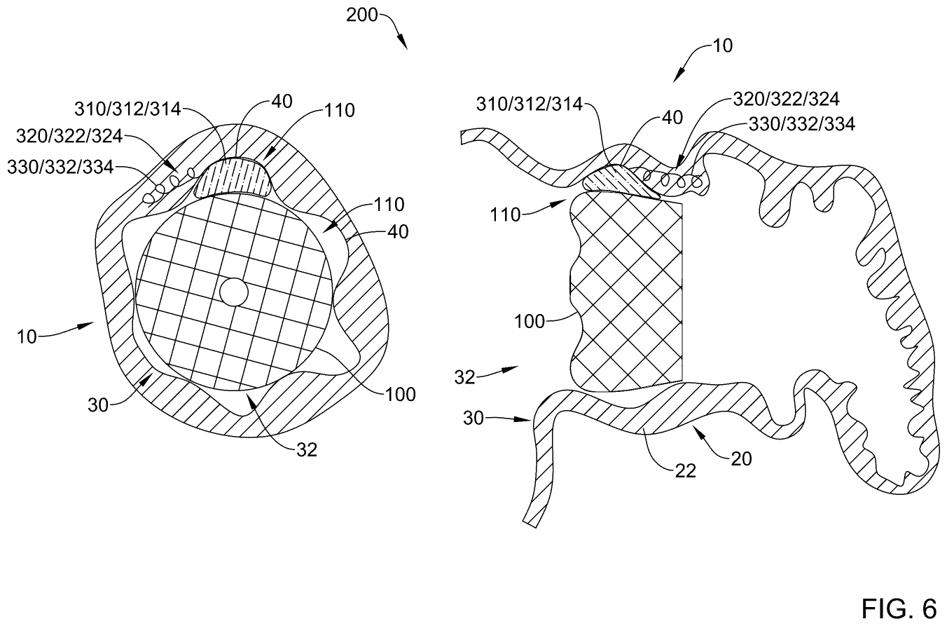

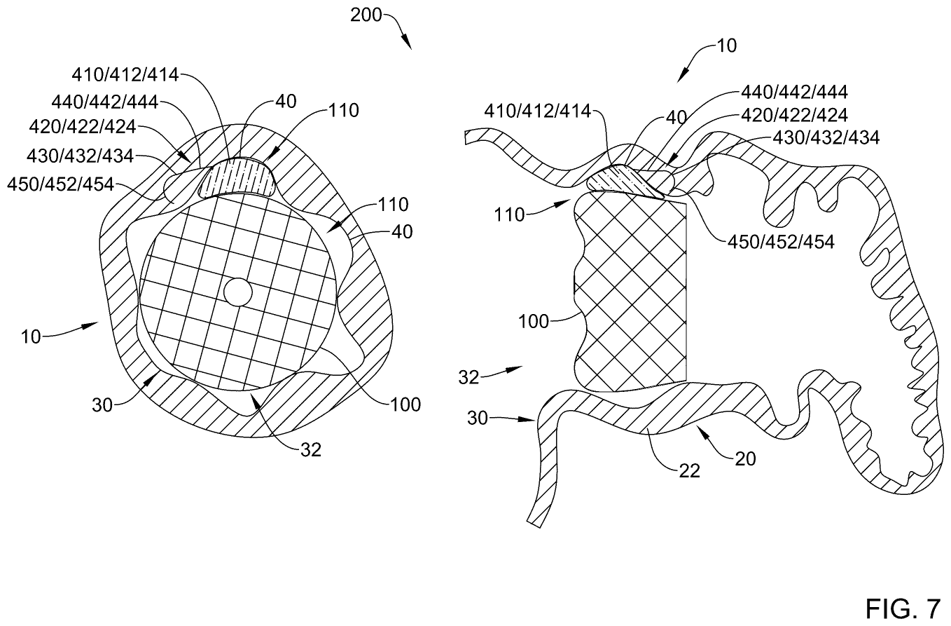

In some embodiments, the anchoring element 320/420, the second anchoring element 322/422, and/or the third anchoring element 324/424 may be configured to engage with and/or pierce tissue of the left atrial appendage 10 (e.g., the side wall and/or the main body 20 of the left atrial appendage 10), as seen in FIGS. 6-7.

In some embodiments, the at least one arm 330/430, the at least one second arm 432, the at least one third arm 434 may be configured to engage with and/or pierce tissue of the left atrial appendage 10 (e.g., the side wall and/or the main body 20 of the left atrial appendage 10). In some embodiments, the helical coil 331, the second helical coil 333, and/or the third helical coil 335 may be configured to engage with and/or pierce tissue of the left atrial appendage 10 (e.g., the side wall and/or the main body 20 of the left atrial appendage 10), as seen in FIG. 6.

In some embodiments, the longitudinally extending portion 440 and/or the laterally extending portion 450, the second longitudinally extending portion 442 and/or the second laterally extending portion 452, and/or the third longitudinally extending portion 444 and/or the third laterally extending portion 454 may be configured to engage with and/or pierce tissue of the left atrial appendage 10 (e.g., the side wall and/or the main body 20 of the left atrial appendage 10), as seen in FIG. 7. Other configurations are also contemplated.

In some embodiments, the piece of expandable foam 210/310/410, the second piece of expandable foam 212/312/412, and/or the third piece of expandable foam 214/314/414 may be configured to remain within the gap 110 or gaps between the occlusive implant 100 and the side wall (e.g., the main body 20) of the left atrial appendage 10 permanently (e.g., the piece of expandable foam 210/310/410, the second piece of expandable foam 212/312/412, and/or the third piece of expandable foam 214/314/414 is never removed from the gap 110 or gaps between the occlusive implant 100 and the side wall (e.g., the main body 20) of the left atrial appendage 10 by the practitioner).

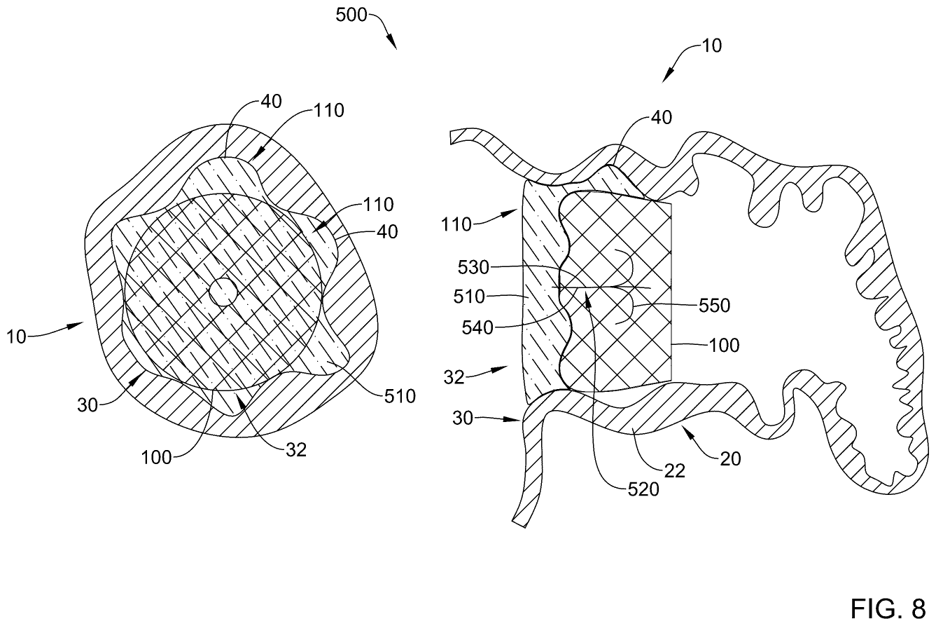

In some embodiments, a gap sealing system 500 for use with an occlusive implant 100 disposed within the left atrial appendage 10 of a patient may comprise a piece of expandable foam 510 configured to expand from a delivery configuration (e.g., similar to FIG. 3) to an expanded configuration (e.g., FIG. 8) in vivo. In embodiments, a kit for use in sealing a gap 110 with an occlusive implant 100 disposed within the left atrial appendage may comprise various shapes, sizes, and configurations of the piece of expandable foam 510, similar to those described herein. In some embodiments, the kit may comprise the piece of expandable foam 510, a second piece of expandable foam, and/or a third piece of expandable foam. The elements and/or features of the gap sealing system 500 discussed herein are not intended to be limiting and/or all-inclusive. The skilled person will recognize that the gap sealing system 500 and/or the kit may comprise fewer elements or more elements than those explicitly described.

In some embodiments, the gap sealing system 500 and/or the kit may comprise different sizes and/or shapes (e.g., different configurations) of the piece of expandable foam 510 which may be selected by the practitioner at the time of use and/or during an implantation procedure according to the desired usage. In some embodiments, the gap sealing system 500 and/or the kit may comprise multiple instances of the piece of expandable foam 510 in a particular size and/or shape. In some embodiments, the gap sealing system 500 and/or the kit may comprise multiple instances of the piece of expandable foam 510 in different sizes and/or shapes. In some embodiments, where the one or more lobes 40 comprises two or more lobes, and/or the two or more lobes are different sizes, more than one piece of expandable foam 510 may be used and/or required.

In at least some embodiments, the piece of expandable foam 510, the second piece of expandable foam, and/or the third piece of expandable foam may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed size. In some examples, the collapsibility ratio of the shape memory polymer and/or the shape memory foam may be at least 5 times, at least 7 times, at least 8 times, at least 9 times, at least 10 times, at least 12 times, or more. In one example, a piece of expandable foam may have an outer diameter of about 32 millimeters in the expanded configuration and about 3 millimeters in the compressed configuration, producing a collapsibility ratio of at least 10 times (e.g., at least 10:1). Other configurations are also contemplated. In at least some embodiments, the piece of expandable foam 510, the second piece of expandable foam, and/or the third piece of expandable foam may be configured as open celled foam.

In some embodiments, the piece of expandable foam 510, the second piece of expandable foam, and/or the third piece of expandable foam and/or the shape memory foam may be formed from a biocompatible material. In at least some embodiments, the piece of expandable foam 510, the second piece of expandable foam, and/or the third piece of expandable foam may be non-biodegradable and/or non-bioabsorbable. In some alternative embodiments, the piece of expandable foam 510, the second piece of expandable foam, and/or the third piece of expandable foam may be biodegradable and/or bioabsorbable over time. In some embodiments, the piece of expandable foam 510, the second piece of expandable foam, and/or the third piece of expandable foam may be configured to promote endothelization and/or tissue ingrowth. In some embodiments, the piece of expandable foam 510, the second piece of expandable foam, and/or the third piece of expandable foam may include a coating, a material, and/or a component that promotes endothelization and/or tissue ingrowth. Other configurations are also contemplated.

In some embodiments, the piece of expandable foam 510, the second piece of expandable foam, and/or the third piece of expandable foam may be configured to be biodegradable and/or bioabsorbable over time. In some embodiments, the piece of expandable foam 510, the second piece of expandable foam, and/or the third piece of expandable foam may be configured to be biodegradable over at least 30 days' time. In some embodiments, the piece of expandable foam 510, the second piece of expandable foam, and/or the third piece of expandable foam may be configured to be biodegradable over at least 60 days' time. In some embodiments, the piece of expandable foam 510, the second piece of expandable foam, and/or the third piece of expandable foam may be configured to be biodegradable over at least 90 days′, 180 days′, or 365 days' time. Other configurations are also contemplated.

In some embodiments, the piece of expandable foam 510, the second piece of expandable foam, and/or the third piece of expandable foam may be configured to prevent thrombus formation (e.g., within the left atrial appendage 10 and/or on the piece of expandable foam 510, the second piece of expandable foam, and/or the third piece of expandable foam). In some embodiments, the piece of expandable foam 510, the second piece of expandable foam, and/or the third piece of expandable foam may include anti-thrombus agent(s) and/or medicament(s). In some embodiments, the piece of expandable foam 510, the second piece of expandable foam, and/or the third piece of expandable foam may be configured to absorb blood and/or bodily fluid(s). In some embodiments, the piece of expandable foam 510, the second piece of expandable foam, and/or the third piece of expandable foam may be configured to trap thrombus. In some embodiments, the piece of expandable foam 510, the second piece of expandable foam, and/or the third piece of expandable foam may be configured to promote tissue ingrowth and/or endothelization. Other configurations are also contemplated.

In some embodiments, the piece of expandable foam 510, the second piece of expandable foam, and/or the third piece of expandable foam may each comprise different sizes and/or shapes in the delivery configuration and/or in the expanded configuration (when unconstrained). In some embodiments, the piece of expandable foam 510, the second piece of expandable foam, and/or the third piece of expandable foam may be generally cylindrical, similar to those shown in FIG. 3. In other examples, the piece of expandable foam 510, the second piece of expandable foam, and/or the third piece of expandable foam may be oblong, the piece of expandable foam 510, the second piece of expandable foam, and/or the third piece of expandable foam may be cubic, spherical, or flattened, etc. Other configurations, including combinations thereof, are also contemplated.

In some embodiments, the piece of expandable foam 510, the second piece of expandable foam, and/or the third piece of expandable foam may be unconstrained by outside forces and/or structure(s) in the delivery configuration. In some embodiments, the piece of expandable foam 510, the second piece of expandable foam, and/or the third piece of expandable foam may be self-maintained in the delivery configuration by shape memory properties. Other configurations are also contemplated.

In some embodiments, the gap sealing system 500 and/or the kit may comprise an anchoring element 520 extending from the piece of expandable foam 510 in the delivery configuration and/or the expanded configuration. In some embodiments, the gap sealing system 500 may comprise a second anchoring element extending from the second piece of expandable foam in the delivery configuration and/or the expanded configuration. In some embodiments, the gap sealing system 500 may comprise a third anchoring element extending from the third piece of expandable foam in the delivery configuration and/or the expanded configuration.

In some embodiments, the anchoring element 520 may be fixedly attached to the piece of expandable foam 510. In some embodiments, the second anchoring element may be fixedly attached to the second piece of expandable foam. In some embodiments, the third anchoring element may be fixedly attached to the third piece of expandable foam. In some embodiments, the anchoring element 520 may be integrally formed with the piece of expandable foam 510. In some embodiments, the second anchoring element may be integrally formed with the second piece of expandable foam. In some embodiments, the third anchoring element may be integrally formed with the third piece of expandable foam. In some embodiments, the anchoring element 520, the second anchoring element, and/or the third anchoring element may be formed from a metallic material, a polymeric material, or from a shape memory material and/or a shape memory polymer. Other configurations are also contemplated.

In some embodiments, the anchoring element 520 may be formed from a different material than the piece of expandable foam 510. In some embodiments, the second anchoring element may be formed from a different material than the second piece of expandable foam. In some embodiments, the third anchoring element may be formed from a different material than the third piece of expandable foam.

In some embodiments, the anchoring element 520 may comprise at least one arm 530 extending distally from the piece of expandable foam 510 in the delivery configuration and/or the expanded configuration. In some embodiments, the at least one arm 530 and/or each arm of the at least one arm 530 may comprise a longitudinally extending portion 540 and a laterally extending portion 550. In some embodiments, the laterally extending portion 550 may extend laterally from a distal end of the longitudinally extending portion 540. In some embodiments, the longitudinally extending portion 540 and the laterally extending portion 550 may form a hook.

In some embodiments, the second anchoring element may comprise at least one second arm extending distally from the second piece of expandable foam in the delivery configuration and/or the expanded configuration. In some embodiments, the at least one second arm and/or each arm of the at least one second arm may comprise a second longitudinally extending portion and a second laterally extending portion. In some embodiments, the second laterally extending portion may extend laterally from a distal end of the second longitudinally extending portion. In some embodiments, the second longitudinally extending portion and the second laterally extending portion may form a second hook.

In some embodiments, the third anchoring element may comprise at least one third arm extending distally from the third piece of expandable foam in the delivery configuration and/or the expanded configuration. In some embodiments, the at least one third arm and/or each arm of the at least one third arm may comprise a third longitudinally extending portion and a third laterally extending portion. In some embodiments, the third laterally extending portion may extend laterally from a distal end of the third longitudinally extending portion. In some embodiments, the third longitudinally extending portion and the third laterally extending portion may form a third hook.

In some embodiments, the at least one arm 530 may comprise a helical coil (not shown) extending distally from the piece of expandable foam 510 in the delivery configuration and/or the expanded configuration, the at least one second arm may comprise a second helical coil extending distally from the second piece of expandable foam in the delivery configuration and/or the expanded configuration, and the at least one third arm may comprise a third helical coil extending distally from the third piece of expandable foam in the delivery configuration and/or the expanded configuration. Other configurations, including combinations thereof, are also contemplated.

In some embodiments, the gap sealing system 500, the kit, and/or the piece of expandable foam 510 may optionally comprise a radiopaque marker (not shown) coupled to the piece of expandable foam 510. In some embodiments, the gap sealing system 500, the kit, and/or the second piece of expandable foam may optionally comprise a second radiopaque marker coupled to the second piece of expandable foam. In some embodiments, the gap sealing system 500, the kit, and/or the third piece of expandable foam may optionally comprise a third radiopaque marker coupled to the third piece of expandable foam.

In some embodiments, the anchoring element 520 may be fixedly attached to the radiopaque marker. In some embodiments, the second anchoring element may be fixedly attached to the second radiopaque marker. In some embodiments, the third anchoring element may be fixedly attached to the third radiopaque marker. Other configurations are also contemplated.

In some embodiments, the radiopaque marker may be embedded within the piece of expandable foam 510. In some embodiments, the second radiopaque marker may be embedded within the second piece of expandable foam. In some embodiments, the third radiopaque marker may be embedded within the third piece of expandable foam. In some embodiments, the radiopaque marker may comprise a radiopaque substance or a radiopaque material disposed within the piece of expandable foam 510 (e.g., the piece of expandable foam 510 may be doped with and/or may include the radiopaque substance or the radiopaque material). In some embodiments, the second radiopaque marker may comprise a radiopaque substance or a radiopaque material disposed within the second piece of expandable foam (e.g., the second piece of expandable foam may be doped with and/or may include the radiopaque substance or the radiopaque material). In some embodiments, the third radiopaque marker may comprise a radiopaque substance or a radiopaque material disposed within the third piece of expandable foam (e.g., the third piece of expandable foam may be doped with and/or may include the radiopaque substance or the radiopaque material).

In some embodiments, the gap sealing system 500 and/or the kit may comprise a tubular member (not shown) sized and configured to deliver the piece of expandable foam 510 to the left atrial appendage 10. In some embodiments, the gap sealing system 500 and/or the kit may comprise a second tubular member sized and configured to deliver the second piece of expandable foam to the left atrial appendage 10. In some embodiments, the gap sealing system 500 and/or the kit may comprise a third tubular member sized and configured to deliver the third piece of expandable foam to the left atrial appendage 10.

In some embodiments, the tubular member, the second tubular member, and/or the third tubular member may be formed from a metallic material, a super-elastic material and/or a linear-elastic material, or a polymeric material. Other configurations are also contemplated. In some alternative embodiments, the tubular member, the second tubular member, and/or the third tubular member may be a tubular member, a catheter, a delivery sheath, etc. (e.g., the use of a tubular member and/or a metallic tubular member is not necessarily required). Other configurations are also contemplated.

In some embodiments, during delivery to the left atrial appendage 10, the tubular member may be adapted and configured to constrain the piece of expandable foam 510 in the delivery configuration. In some embodiments, during delivery to the left atrial appendage 10, the second tubular member may be adapted and configured to constrain the second piece of expandable foam in the delivery configuration. In some embodiments, during delivery to the left atrial appendage 10, the third tubular member may be adapted and configured to constrain the third piece of expandable foam in the delivery configuration.

In some embodiments, the piece of expandable foam 510, the second piece of expandable foam, and/or the third piece of expandable foam may be configured to shift from the delivery configuration toward and/or to the expanded configuration when unconstrained. In some embodiments, the piece of expandable foam 510, the second piece of expandable foam, and/or the third piece of expandable foam may be configured to shift from the delivery configuration toward and/or to the expanded configuration when unconstrained by the tubular member, the second tubular member, and/or the third tubular member, respectively. In some embodiments, the piece of expandable foam 510, the second piece of expandable foam, and/or the third piece of expandable foam may be configured to shift from the delivery configuration toward and/or to the expanded configuration under predetermined conditions (e.g., at a preselected temperature, when exposed to a preselected fluid or amount of fluid, etc.).

In some embodiments, the piece of expandable foam 510, the second piece of expandable foam, and/or the third piece of expandable foam may be configured to be disposed proximal of the occlusive implant 100 (to occlude the ostium 30 of the left atrial appendage 10). In some embodiments, the piece of expandable foam 510, the second piece of expandable foam, and/or the third piece of expandable foam may be further configured to be extend alongside the occlusive implant 100 to occlude and/or to fill a gap 110 or gaps around the margin(s) and/or the periphery of the occlusive implant 100 between the occlusive implant 100 and the side wall (e.g., the main body 20) of the left atrial appendage 10.

In some embodiments, the piece of expandable foam 510, the second piece of expandable foam, and/or the third piece of expandable foam may be configured to adapt and conform to surrounding anatomy and/or structure(s) (e.g., the occlusive implant 100) when shifting from the delivery configuration toward and/or to the expanded configuration in vivo.

In some embodiments, the piece of expandable foam 510, the second piece of expandable foam, and/or the third piece of expandable foam may be not directly attachable to the occlusive implant 100. In some embodiments, the piece of expandable foam 510, the second piece of expandable foam, and/or the third piece of expandable foam may be configured to engage with and/or extend into an expandable framework of the occlusive implant 100. Other configurations are also contemplated.

In some embodiments, the anchoring element 520, the second anchoring element, and/or the third anchoring element may be configured to extend into an interior of the occlusive implant 100 and/or the expandable framework of the occlusive implant 100 in vivo, and/or to engage with the occlusive implant 100 and/or the expandable framework of the occlusive implant 100 within the left atrial appendage 10 in vivo, as seen in FIG. 8.

In some embodiments, the at least one arm 530, the at least one second arm, the at least one third arm may be configured to engage with the occlusive implant 100 and/or the expandable framework of the occlusive implant 100 within the left atrial appendage 10. In some embodiments, the helical coil, the second helical coil, and/or the third helical coil may be configured to engage with the occlusive implant 100 and/or the expandable framework of the occlusive implant 100 within the left atrial appendage 10.

In some embodiments, the longitudinally extending portion 540 and/or the laterally extending portion 550, the second longitudinally extending portion and/or the second laterally extending portion, and/or the third longitudinally extending portion and/or the third laterally extending portion may be configured to engage with the occlusive implant 100 and/or the expandable framework of the occlusive implant 100 within the left atrial appendage 10. In some embodiments, the laterally extending portion 550 of the at least one arm 530, the second laterally extending portion of the at least one second arm, and/or the third laterally extending portion of the at least one third arm may be configured to extend into an interior of the occlusive implant 100 and/or the expandable framework of the occlusive implant 100 in vivo to anchor the piece of expandable foam 510, the second piece of expandable foam, and/or the third piece of expandable foam to the occlusive implant 100 within the left atrial appendage 10, as seen in FIG. 8. Other configurations are also contemplated.

In some alternative embodiments, the anchoring element 520, the second anchoring element, and/or the third anchoring element may be configured to engage with and/or pierce tissue of the left atrial appendage 10 (e.g., the side wall and/or the main body 20 of the left atrial appendage 10). In some embodiments, the longitudinally extending portion 540 and/or the laterally extending portion 550, the second longitudinally extending portion and/or the second laterally extending portion, and/or the third longitudinally extending portion and/or the third laterally extending portion may be configured to engage with and/or pierce tissue of the left atrial appendage 10 (e.g., the side wall and/or the main body of the left atrial appendage 10). Other configurations are also contemplated.

In some embodiments, the piece of expandable foam 510, the second piece of expandable foam, and/or the third piece of expandable foam may be configured to remain within the ostium 30 of the left atrial appendage 10, and/or the gap 110 or gaps between the occlusive implant 100 and the side wall (e.g., the main body 20) of the left atrial appendage 10, permanently (e.g., the piece of expandable foam 510, the second piece of expandable foam, and/or the third piece of expandable foam is never removed from the ostium 30 of the left atrial appendage 10, and/or gap 110 or gaps between the occlusive implant 100 and the side wall (e.g., the main body 20) of the left atrial appendage 10, by the practitioner).

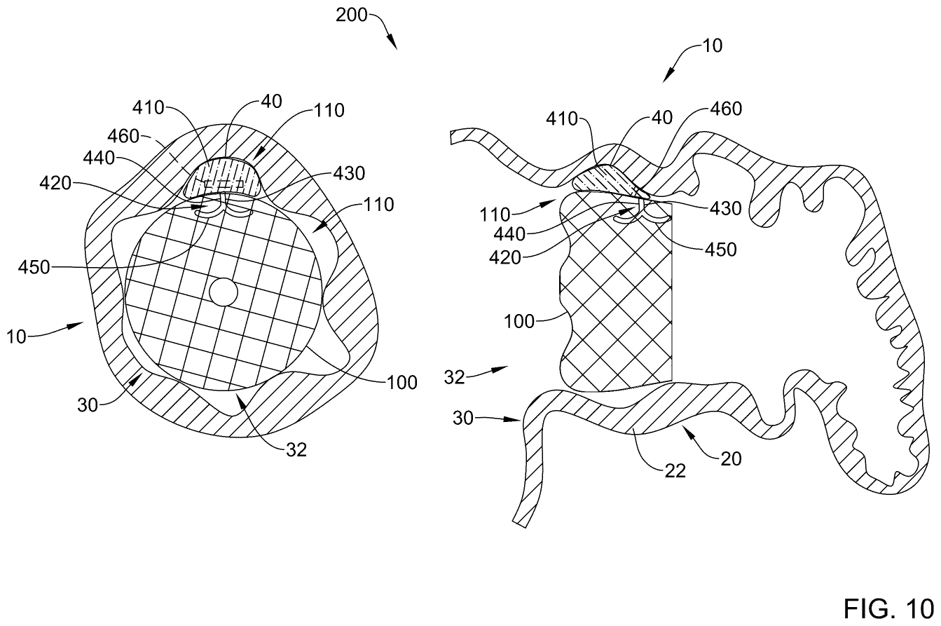

FIGS. 9-10 illustrates selected aspects of the gap sealing system 200 and/or the kit for use in sealing a gap 110 with an occlusive implant 100 disposed within the left atrial appendage 10. For the purpose of discussion, the anchoring element 420 and elements and/or components thereof are illustrated and discussed. However, it shall be understood that the illustration and discussion also applies to the second anchoring element 422 and/or the third anchoring element 424 and the elements and/or components thereof.

As discussed herein, in some embodiments, the anchoring element 420 may be formed from a shape memory material and/or a shape memory polymer. In some embodiments, the anchoring element 420 may comprise at least one arm 430 extending distally from the piece of expandable foam 410 in the delivery configuration and/or the expanded configuration. In some embodiments, the at least one arm 430 (e.g., FIG. 10) and/or each arm of the at least one arm 430 may comprise a longitudinally extending portion 440 (e.g., FIG. 10) and a laterally extending portion 450 (e.g., FIG. 10). In some embodiments, the laterally extending portion 450 may extend laterally from a distal end of the longitudinally extending portion 440. In some embodiments, the longitudinally extending portion 440 and the laterally extending portion 450 may form a hook.

In some embodiments, the anchoring element 420 may be configured to shift between a delivery configuration (e.g., FIG. 9) and an expanded configuration (e.g., FIG. 10) in vivo. In the delivery configuration, the anchoring element 420 may assume and/or may have an atraumatic shape.

In some embodiments, the piece of expandable foam 410 may be configured to adapt and conform to surrounding anatomy and/or structure(s) (e.g., the occlusive implant 100) when shifting from the delivery configuration toward and/or to the expanded configuration in vivo. In some embodiments, the piece of expandable foam 410 may be not directly attachable to the occlusive implant 100. In some embodiments, the piece of expandable foam 410 may be configured to engage with and/or extend into an expandable framework of the occlusive implant 100. Other configurations are also contemplated.

In the expanded configuration, the anchoring element 420 may be configured to engage with and/or entangle with the occlusive implant 100 and/or the expandable framework of the occlusive implant 100 within the left atrial appendage 10, as seen in FIG. 10. In some embodiments, the at least one arm 430 may be configured to engage with the occlusive implant 100 and/or the expandable framework of the occlusive implant 100 within the left atrial appendage 10. In some embodiments, the longitudinally extending portion 440 and/or the laterally extending portion 450 may be configured to engage with the occlusive implant 100 and/or the expandable framework of the occlusive implant 100 within the left atrial appendage 10. In some embodiments, the laterally extending portion 450 of the at least one arm 430 may be configured to extend into an interior of the occlusive implant 100 and/or the expandable framework of the occlusive implant 100 in vivo to anchor the piece of expandable foam 410 to the occlusive implant 100 within the left atrial appendage 10, as seen in FIG. 10. Other configurations are also contemplated.

In at least some embodiments, the anchoring element 420, the at least one arm 430, the longitudinally extending portion 440, and/or the laterally extending portion 450 may be adapted and configured to avoid piercing and/or penetrating tissue of the left atrial appendage 10. For example, the physical properties (e.g., hardness/softness, flexibility, etc.) of the anchoring element 420, the at least one arm 430, the longitudinally extending portion 440, and/or the laterally extending portion 450 may be insufficient to pierce and/or penetrate tissue of the left atrial appendage 10, thereby avoiding tissue damage. Other configurations are also contemplated.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the piece of expandable foam, the anchoring element, the at least one arm, the radiopaque marker, the tubular member, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co —Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A gap sealing system for use with an occlusive implant disposed within a left atrial appendage of a patient, comprising:
a piece of expandable foam configured to expand from a delivery configuration to an expanded configuration in vivo; and
an anchoring element extending from the piece of expandable foam.

2. The gap sealing system of claim 1, further comprising a radiopaque marker coupled to the piece of expandable foam.

3. The gap sealing system of claim 2, wherein the anchoring element is fixedly attached to the radiopaque marker.

4. The gap sealing system of claim 1, wherein the anchoring element is integrally formed with the piece of expandable foam.

5. The gap sealing system of claim 1, wherein the anchoring element is formed from a different material than the piece of expandable foam.

6. The gap sealing system of claim 5, wherein the anchoring element is formed from a metallic material.

7. The gap sealing system of claim 1, further comprising a tubular member sized and configured to deliver the piece of expandable foam to the left atrial appendage.

8. The gap sealing system of claim 1, wherein the anchoring element is configured to engage with the occlusive implant.

9. The gap sealing system of claim 1, wherein the anchoring element is configured to pierce tissue of the left atrial appendage.

10. The gap sealing system of claim 1, wherein the piece of expandable foam is formed from a shape memory polymer.

11. The gap sealing system of claim 1, wherein the piece of expandable foam is configured to be disposed alongside the occlusive implant to occlude a gap between the occlusive implant and a side wall of the left atrial appendage.

12. A gap sealing system for use with an occlusive implant disposed within a left atrial appendage of a patient, comprising:
a piece of expandable foam configured to expand from a delivery configuration to an expanded configuration in vivo; and
an anchoring element comprising at least one arm extending distally from the piece of expandable foam in the expanded configuration, wherein the at least one arm comprises a longitudinally extending portion and a laterally extending portion;
wherein the piece of expandable foam is configured to be disposed proximal of the occlusive implant to occlude an ostium of the left atrial appendage;
wherein the laterally extending portion of the at least one arm is configured to extend into an interior of the occlusive implant in vivo to anchor the piece of expandable foam to the occlusive implant.

13. The gap sealing system of claim 12, wherein the laterally extending portion extends laterally from a distal end of the longitudinally extending portion.

14. The gap sealing system of claim 12, wherein the piece of expandable foam is not directly attachable to the occlusive implant.

15. A kit for use in sealing a gap with an occlusive implant disposed within a left atrial appendage of a patient, comprising:
a piece of expandable foam, wherein the piece of expandable foam is configured to expand from a delivery configuration to an expanded configuration in vivo;
a second piece of expandable foam, wherein the second piece of expandable foam is configured to expand from a delivery configuration to an expanded configuration in vivo;
wherein the piece of expandable foam has a first overall volume, and the second piece of expandable foam has a second overall volume different from the first overall volume;
a tubular member sized and configured to deliver the piece of expandable foam to the left atrial appendage; and
a second tubular member sized and configured to deliver the second piece of expandable foam to the left atrial appendage.

16. The kit of claim 15, wherein the piece of expandable foam and the second piece of expandable foam are each configured to be disposed alongside the occlusive implant to fill a gap between the occlusive implant and a side wall of the left atrial appendage.

17. The kit of claim 15, wherein the piece of expandable foam comprises an anchoring element extending distally from the piece of expandable foam; and
wherein the second piece of expandable foam comprises a second anchoring element extending distally from the second piece of expandable foam.

18. The kit of claim 17, wherein the anchoring element is formed from a shape memory polymer.

19. The kit of claim 17, wherein the anchoring element is formed from a metallic material.

20. The kit of claim 30, further comprising a third piece of expandable foam, wherein the third piece of expandable foam is configured to expand from a delivery configuration to an expanded configuration in vivo, the third piece of expandable foam having a third overall volume different from the first overall volume and the second overall volume; and
a third tubular member sized and configured to deliver the third piece of expandable foam to the left atrial appendage.
